(19) United States
(12) Patent Application Publication
(10) Pub. No.: US 2002/0115721 A1
(43) Pub. Date: Aug. 22, 2002

(54) Esters of alkycarboxy amino acids as prodrugs of modulators of the kainate receptor

(75) Inventors: Xue-Feng Pei, Lansdale, PA (US); Maria-Luisa Maccecchini, West Chester, PA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Annovis, Inc.

(21) Appl. No.: 10/020,842

(22) Filed: Oct. 30, 2001

Related U.S. Application Data
(60) Provisional application No. 60/244,411, filed on Oct. 30, 2000.

(51) Int. Cl. A61K 31/221; C07C 229/24; C07C 229/32

(52) U.S. Cl. 514/566; 514/551; 560/171; 560/125

(57) ABSTRACT

Disclosed are prodrug compounds of a class of alkyl carboxy amino acid analogs of glutamic acid that act as specific regulators of the kainate EAA receptor cation channel. These compounds are useful for treating neurological, neuropsychological, neuropsychiatric, neurodegenerative, neuropsychopharmacological and functional disorders associated with excessive or insufficient activation of the kainate subtype of the ionotropic EAA receptors; treating cognitive disorders associated with deactivation, suboptimal activation or over-activation of the kainate receptor; alleviating pain and improving and enhancing memory, learning, and associated mental processes.

### CROSS REFERENCE TO RELATED APPLICATIONS

Priority is claimed to U.S. Provisional application Ser. No. 60/244,411, filed Oct. 30, 2000, the teachings of which are incorporated herein.

### BACKGROUND OF THE INVENTION

This invention generally relates to the kainate subtype of the postsynaptic glutamate receptor and more specifically to prodrugs of compounds other than kainate which bind to the kainate receptor and methods of use thereof.

During the past twenty-five years, a revolution in understanding the basic structure and chemistry of the synaptic interconnections of neural tissues has taken place, which has yielded knowledge relevant to the treatment of neural tissue damage and disorders. The studies have centered around an understanding of the properties of the neurochemical transmitters released from presynaptic membranes and, most importantly, the postsynaptic receptors for these transmitters. During the past ten years, a great deal of attention has been directed to the excitatory amino acids (EAAs), principally glutamic acid (the primary excitatory neurotransmitter) and aspartic acid, and their receptors since these amino acids mediate the fast excitatory transmission in the mammalian central nervous system. Thus, glutamic acid can bring about changes in the postsynaptic neuron that reflect the strength of the incoming neural signals.

Two major classes of EAA receptors are distinguished: ionotropic and metabotropic. The ionotropic receptors contain ligand-gated ion channels and mediate ion fluxes for signaling, while the metabotropic receptors use G-proteins for signaling. Further sub-classification of the ionotropic EAA glutamate receptors is based upon the agonists (stimulating agents) other than glutamic and aspartic acid that selectively activate the receptors. Presently, it is believed that there are three major subtypes of ionotropic glutamate receptors based on binding at defined concentrations: 1) a receptor responsive to N-methyl-D-aspartate (NMDA); 2) a receptor not responsive to NMDA but responsive to α-amino-3-hydroxy-5-methyl-4-isoxazolepropionic acid (AMPA); and 3) a receptor not responsive to NMDA but responsive to kainate. The NMDA receptor controls the flow of both divalent (Ca⁺⁺) and monovalent (Na⁺, K⁺) ions into the postsynaptic neural cell although it is the Ca⁺⁺ flux which is of the greatest interest. The AMPA and kainate receptors also regulate the flow into postsynaptic cells of monovalent K⁺ and Na⁺ and occasionally divalent Ca⁺⁺.

EAA receptors have been implicated during development in specifying neuronal architecture and synaptic connectivity and may be involved in experience-dependent synaptic modifications. These receptors have also drawn interest since they appear to be involved in a broad spectrum of CNS disorders. For example, during brain ischemia caused by stroke or traumatic injury, excessive amounts of the EAA glutamic acid are released from damaged or oxygen deprived neurons. Binding of this excess glutamic acid to the postsynaptic glutamate receptors opens their ligand-gated ion channels, thereby allowing an ion influx which in turn activates a biochemical cascade resulting in protein, nucleic acid and lipid degradation and cell death. This phenomenon, known as excitotoxicity, is also thought to be responsible for the neurological damage associated with other disorders ranging from hypoglycemia, ischemia, and epilepsy to chronic neurodegeneration in Huntington's, Parkinson's, and Alzheimer's diseases.

Drugs acting on the ionotropic EAA receptors are, therefore, expected to have enormous therapeutic potential. U.S. Pat. No. 4,904,681 to Cordi, et al., discloses the use of a compound, D-cycloserine, which modulates the NMDA receptor to improve/enhance memory and to treat cognitive deficits linked to a neurological disorder. U.S. Pat. No. 5,061,721 to Cordi et al. discloses the use of a combination of D-cycloserine and D-alanine to treat Alzheimer's disease, age-associated memory impairment, learning deficits, and psychotic disorders, as well as to improve memory or learning in healthy individuals. U.S. Pat. No. 5,086,072 to Trullas et al. discloses the use of another compound, 1-aminocyclo-propane carboxylic acid (ACPC), which modulates the NMDA receptor, to treat mood disorders including major depression, bipolar disorder, dysthymia and seasonal affective disorder. Trullas et al., also discloses that ACPC mimics the actions of clinically effective antidepressants in animal models, and that ACPC and its derivatives may be used to treat neuropharmacological disorders resulting from excessive activation of the NMDA receptor.

The EAA receptors are also involved with the physiological basis for drug addiction. U.S. Pat. No. 5,523,323 to Maccecchini has described not only tolerance to, but also dependence on, opiates which can be prevented by a partial agonist of the NMDA receptor. Presently, it is believed that a balance in the activities of the three types of EAA ionotropic receptors may be necessary to achieve normal neurological synaptic control. It is known that, in the presence of excess glutamic acid, antagonists of the NMDA receptor prevent immediate excitotoxicity. However, over a longer period of time, all cell death is not completely prevented, which may be due to the excitotoxicity caused by the continued action of the EAAs on the AMPA and kainate receptors.

NMDA, AMPA, and kainate are glutamic acid analogs as shown by the following schematics.

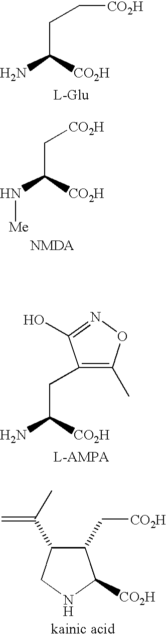

It is remarkable that these analogs can distinguish between receptor types and must reflect subtle differences in the three dimensional conformation of the various binding sites. Selective binding of conformationally restricted analogs suggests that glutamic acid may bind to each receptor in a distinct conformation. Glutamic acid itself has at least nine low energy staggered conformations. The existence of these distinctions also suggests a fine degree of chemical regulation exercised over the EAA receptors and the potential to find selective modulators of the receptors if the necessary binding conformations were understood for each receptor.

Originally isolated from the seaweed Digenea simplex, which grows off the coast of Japan, kainate is a glutamic acid analog having three asymmetric carbon atoms. It is one of the most potent commonly used exogenous excitotoxins, and studies have shown that its neurotoxic action is mediated through the AMPA and kainate receptors. Of particular interest is the fact that the neuronal degeneration caused by kainate excitotoxicity differs significantly from that observed with other EAA receptor agonists. In fact, the degeneration seen in the brains of test animals after kainate exposure is remarkably similar to that seen in the neurodegenerative disorder, Huntington's disease, and temporal lobe epilepsy.

While a great deal has been learned about the regulation of the NMDA receptor, much less is known about the AMPA and kainate receptors. A principal reason for this lack of knowledge is that no compounds are known which selectively modulate the kainate receptor. For example, 6-nitro-7-sulfamoylbenzoquinoxaline-2,3-dione (NBQX) has been reported by Jacobsen, et al., in U.S. Pat. No. 4,889,855 to be an AMPA/kainate receptor antagonist useful for treating neurodegenerative diseases. NBQX is the most AMPA/kainate receptor selective member of the quinoxaline-2,3-dione family of compounds. Though NBQX competitively inhibits glutamate binding to both the AMPA and the kainate receptors, it has an affinity thirty times greater for the AMPA receptor as compared to the kainate receptor and also non-specifically binds to the glycine site of the NMDA receptor. Unfortunately, since NBQX has very limited solubility in water it has not been developed for human use. Thus, it has been impossible to study the effect of the EAA's on kainate receptors without having an unknown contribution from the other receptors. In addition, it has been impossible to selectively prevent damage caused by excess EAA stimulation of the kainate receptor. Clearly, the availability of compounds which selective modulate the kainate receptor could prevent excitotoxic cell death due to excess stimulation.

Since the discovery that glutamic acid and aspartic acid are natural neurotransmitters that activate neuroreceptors, chemists and pharmacologists have attempted to understand the critical aspects of shape, pharmacophore position and pharmacophore type that are important for agonist or antagonist modulation of the receptors. Generally random screening and hit or miss synthesis and testing were used to find new agonists or antagonists for the receptors. It is likely that each glutamic acid receptor subtype, such as NMDA, AMPA or kainate, will have its own requirements for agonist shape and pharmacophore positions along with different shape and pharmacophore positions for antagonists. The optimal way to design new agonists or antagonists is to have an agonist or antagonist model for each receptor subtype that contains the specific shape and pharmacophore positions and then to use this model to link the pharmacophores into novel molecules.

Since glutamic acid is involved in many different biochemical reactions throughout the cell, attempts have been made to find glutamic acid analogs in which the stereochemistry about the various glutamic acid carbons has been altered in an attempt to find other molecules which would have the correct dimensional fit to participate in the biochemical reactions.

Several publications disclose substituted alkyl glutamic acids. H. J. Overman et al (Neuroscience, 26(1), 17-31, 1988, Table 4) describe racemic DL 4-methylglutamic acid, DL 4-fluoroglutamic acid or DL 3-methylaspartic acid and their binding to the NMDA receptor. Overman teaches that the methyl group is detrimental to NMDA binding affinity compared to glutamic acid itself.

Much of what has been learned about the NMDA receptor has been made possible by the discovery of compounds which block one or another action of the various modulatory agents. The approach of using blocking agents to map pathways has a long history in biochemical and biophysical research and very often these blocking agents have been discovered to be useful therapeutic agents. Compounds which selectively bind to the kainate receptor are no exception.

Alkylcarboxy amino acid compounds that can modulate the kainate receptor are disclosed in U.S. Pat. No. 5,731,348 to Gu. These compounds are useful for treating neurological, neuropsychological, neuropsychiatric, neurodegenerative, neuropsychopharmacological and functional disorders associated with excessive or insufficient activation of the kainate subtype of the ionotropic EAA receptors; treating cognitive disorders associated with deactivation, suboptimal activation or over-activation of the kainate receptor; alleviating pain and improving and enhancing memory, learning, and associated mental processes. It would be useful to increase the bioavailability, membrane permeability, and half life of these compounds.

It is therefore an object of the present invention to provide prodrugs of compounds which selectively bind to the kainate receptor.

It is a further object of the present invention to provide prodrugs of compounds which selectively modulate or regulate the kainate receptor function.

It is a further object of this invention to provide compounds and methods for use of the compounds to specifically regulate the flow of cations through the kainate ligand-gated EAA receptor complex.

It is still another object of the present invention to provide compounds and methods of use thereof for treatment of neurological, neuropsychological, neuropsychiatric and neuropsychopharmocological conditions, neurodegeneration after central nervous system or spinal trauma and injury, alleviation of pain, and enhancement of learning and memory.

A further object of the invention is to provide compounds and methods for use thereof to treat chemical toxicity in patients using compounds which selectively act at the kainate receptor.

### BRIEF SUMMARY OF THE INVENTION

Prodrugs of a class of alkyl carboxy amino acid compounds have been discovered which are converted to the alkyl carboxy amino acid compounds in plasma and which in turn bind to the kainate receptor and modulate the kainate receptor function. Illustrative prodrug compounds include:

(2S,4R)-4-methyl glutamic acid dimethyl ester;

(2S,4R)-4-methyl glutamic acid diethyl ester;

(2S,4R)-4-methyl glutamic acid di-tert-butyl ester;

(2R,4S)-4-methyl glutamic acid dimethyl ester;

(2R,4S)-4-methyl glutamic acid diethyl ester;

(2R,4S)-4-methyl glutamic acid di-tert-butyl ester.

These compounds, in combination with suitable pharmaceutically acceptable carriers, are useful in methods to treat: 1) neurological, neuropsychological, neuropsychiatric, neurodegenerative, neuropsychopharmacological and functional disorders associated with excessive or insufficient activation of the kainate subtype of the ionotropic EAA receptors; 2) cognitive disorders associated with deactivation, suboptimal activation, or over-activation of the kainate receptor; 3) to improve and enhance memory, learning, and associated mental processes; and 4) alleviation of pain.

### BRIEF DESCRIPTION OF THE DRAWING

FIG. 1 illustrates the synthesis of (2S,4R)-4-methyl glutamic acid di-tert-butyl ester.

### DETAILED DESCRIPTION OF THE INVENTION

I. Glossary of Terms.

The term “agonist” as used herein means any compound which increases the flow of cations through the Kainate receptor, that is, works as a channel opener, and which has not been observed to decrease the flow of cations through the same receptor.

The term “antagonist” as used herein means any compound which reduces the flow of cations through the Kainate receptor, that is, works as a channel closer, and which has not been observed to increase the flow of cations through the same receptor.

The term “partial agonist” as used herein means a compound which modulates an EAA receptor so as to increase or decrease the flux of cations through the ligand-gated channel depending on the presence or absence of the principal site modulator(s). In the absence of the principal site modulator(s), a partial agonist increases the flow of cations through the ligand-gated channel but at a lower flux than achieved by the principal site modulator(s). A partial agonist partially opens the receptor channel. In the presence of the principal site modulator(s), a partial agonist decreases the flow of cations through the ligand-gated channel below the flux normally achieved by the principal site modulator(s).

The term “principal site ligand” as used herein refers to known endogenous ligands binding to a site.

The term “glutamic acid” as used herein means the amino acid L-glutamic acid (“Glu”).

The term “neuropsychopharmacological disorder” as used herein means a disorder resulting from, or associated with, a reduced or excessive flux of ions through the kainate receptor ligand-gated cation channel, and includes cognitive, learning, and memory deficits, chemical toxicity (including substance tolerance and addiction), excitotoxicity, neurodegenerative disorder (such as Huntington's disease, Parkinson's disease, and Alzheimer's disease), post-stroke sequelae, epilepsy, seizures, mood disorders (such as bipolar disorder, dysthymia, and seasonal effective disorder), and depression. Neurodegenerative disorders can result from dysfunction or malfunction of the receptor. As used herein, this term includes pain.

The term “NMDA receptor” as used herein means a postsynaptic receptor which is stimulated, at a minimum, by the EAA glutamic acid as well as by NMDA, but is not stimulated by AMPA or kainate. It is a ligand-gated receptor.

The term “AMPA receptor” as used herein means a postsynaptic receptor which is stimulated by the EAAs glutamic acid as well as by AMPA, but is not stimulated by NMDA and only minimally and at high concentrations by kainate. It is a ligand-gated receptor.

The term “kainate receptor” as used herein means a postsynaptic receptor which is stimulated, at a minimum, by the EAA glutamic acid as well as by kainate, but is not stimulated by NMDA and only minimally and at high concentrations by AMPA. It is a ligand-gated receptor.

The term “potency” as used herein refers to the molar concentration at which a specified effect on a receptor channel is observed. Specifically, potency for a compound exhibiting antagonistic effect is presented as the IC₅₀ value, which is the concentration at which inhibition of channel opening is 50% of the maximum inhibition achievable. Lower values indicate higher potency. Potency for a compound exhibiting agonistic effect is presented as the EC₅₀ value, which is the concentration at which enhancement of channel opening is 50% that of the maximum enhancement achievable. Lower values indicate higher potency.

The term “efficacious” as used herein refers to a comparison of the maximum channel opening or closing and the associated ion flow achieved by a particular compound with maximum channel opening or closing achieved by a principal site ligand. Efficacy refers to magnitude of a specified effect.

The term “pharmacophore” as used herein means an atom or group of atoms that electrostatically or through hydrogen bonds interacts directly with the receptor protein.

The term “specifically binds” as used herein means a compound binding to a receptor with an affinity at least three times as great as a compound which binds to multiple sites or receptors.

The term “prodrug” as used herein means a compound that is converted into a bioactive form in an animal and human body. The prodrug itself may or may not have a bioactivity.

When an alkyl substituent is identified herein, the normal alkyl structure is intended (i.e. butyl is n-butyl) unless otherwise specified. However, when radicals are identified (e.g. R₁), both branched and straight chains are included in the definition of alkyl, alkenyl, and alkynyl.

Pharmaceutically acceptable salts include both the metallic (inorganic) salts and organic salts; a list of which is given in Remington's Pharmaceutical Sciences 17th Edition, p. 1418 (1985). It is well known to one skilled in the art that an appropriate salt form is chosen based on physical and chemical stability, flowability, hydroscopicity and solubility.

Depending on the required activity, the disclosed compounds may be used as pharmaceutical neuroprotectants to treat acute cases of CNS injury and trauma as well as to treat convulsions, mood disorders, alleviation of pain, and other neuropsychiatric and neurodegenerative diseases due, in part, to chronic disturbances in the control of the ion flux through the kainate receptor. Similarly, the disclosed compounds can be selected for the required activity to treat the disorder. As used herein, the common definitions of neuropsychiatric and neurodegenerative disorders are intended, where diagnosis is based on the alleviation of abnormal behavior, rather than histopathology.

II. Synthesis

Ester forms of a class of alkyl carboxy amino acid compounds has been discovered which act as prodrugs. The alkyl carboxy amino acid compounds, when formed, bind to the kainate receptor and modulate the kainate receptor function. The prodrug compounds have the following formula:

An alkyl carboxy amino acid ester compound having the following formula I:

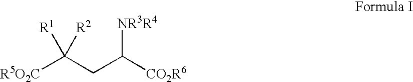

wherein:

R¹, R², R⁵ and R⁶ are independently

1) C1-C6-alkyl,

2) C3-C4-alkenyl,

3) C3-C5-cycloalkyl,

R³ and R⁴ are independently

1) H

2) C1-C6-alkyl,

3) C3-C4-alkenyl,

4) C3-C5-cycloalkyl,

5) C1-C6-alkyl-CO—

6) C1-C6-alkyl-OCO—

7) C1-C6-alkyl-NHCO—

8) HCO—, or

9) C3-C6-alkynyl;

R³ and R⁴ taken together can be —CH₂(CH₂)ₙCH₂—;

n is 0-3;

and pharmaceutically acceptable salts of these compounds.

Preferred compounds are compounds of Formula I wherein:

R¹ is CH₃, R² is H;

R⁵ and R⁶ are independently

1) C1-C6-alkyl,

2) C3-C4-alkenyl, or

3) C3-C5-cycloalkyl;

R³ and R⁴ are independently

1) H,

2) C1-C6-alkyl,

3) C3-C4-alkenyl,

4) C3-C5-cycloalkyl,

5) C1-C6-alkyl-CO—,

6) C1-C6-alkyl-OCO—,

7) C1-C6-alkyl-NHCO—, or

8) HCO—;

R³ and R⁴ taken together can be —CH₂(CH₂)ₙCH₂—;

n is 0-3;

and pharmaceutically acceptable salts of these compounds.

Specifically preferred compounds are:

(2S,4R)-4-methyl glutamic acid dimethyl ester;

(2S,4R)-4-methyl glutamic acid diethyl ester;

(2S,4R)-4-methyl glutamic acid di-tert-butyl ester;

and pharmaceutically acceptable salts of these compounds.

Illustrative compounds include:

(2S,4R)-4-methyl glutamic acid dimethyl ester;

(2S,4R)-4-methyl glutamic acid diethyl ester;

(2S,4R)-4-methyl glutamic acid di-tert-butyl ester;

(2R,4S)-4-methyl glutamic acid dimethyl ester;

(2R,4S)-4-methyl glutamic acid diethyl ester;

(2R,4S)-4-methyl glutamic acid di-tert-butyl ester.

The compounds of Formula I may be prepared using synthetic reactions and techniques available in the art, as described, for example in March, “Advanced Organic Chemistry,” ₄^{th} Edition, 1992, Wiley-Interscience Publication, New York. The reactions are performed in solvents suitable to the reagents and materials employed and suitable for the transformation being effected. Depending upon the synthetic route selected, and the functionality of the starting material or intermediates, the appropriate protection groups and deprotection conditions available in the art of organic synthesis may be utilized in the synthesis of the compound. It is understood by those skilled in the art of organic synthesis that the functionality present on the molecule must be consistent with the chemical transformations proposed. This will frequently necessitate judgment as to the order of synthetic steps, protecting groups required, deprotection conditions and generation of enolate to enable attachment of appropriate groups on the molecule.

The compounds of Formula I where R⁵ and R⁶ are methyl or ethyl groups can be made directly from corresponding amino acids with treatment of a halogenating agent such as thionyl chloride in an alcoholic solvent such as methanol or ethanol at an ambient temperature such as room temperature (Z.-Q. Gu, U.S. Pat. No. 5,731,348). For the preparation of higher alkyl esters, the amino group in the corresponding amino acids shall be protected before the esterification reaction. After esterification, the protecting group is then removed to generate the final ester product.

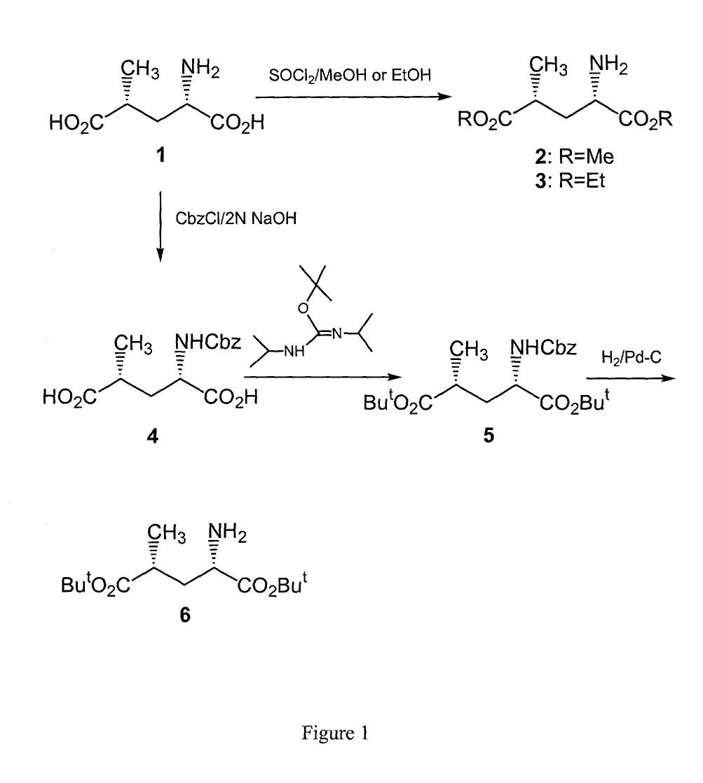

In one embodiment, compounds of Formula I may be synthesized as outlined in FIG. 1.

In FIG. 1, (2S,4R)-glutamic acid (1) is esterified under standard conditions with methanol or ethanol to generate (2S,4R)-glutamic acid esters (2) and (3). For the synthesis of higher alkyl or alkenyl esters, the amine group of 1 is protected under standard conditions (Buehler and Pearson, “Survey of Organic Synthesis”, 1970, Wiley-Interscience Publication, New York) by an appropriate amine protecting group, such as an aromatic amide such as nitrobenzoyl, naphthoyl, N-tert-butoxycarbonyl (BOC) or carbobenzoyl (CBZ). For example, 1 reacts with carbobenzoyl chloride in the presence of appropriate molar equivalents of a base such as NaOH to generate the carbobenzoyl group protected (2S,4R)-glutamic acid 4. 4 is then subjected to esterification reaction with an appropriate esterification agent such as O-tert-butyl-N,N′-diisopropylisourea to generate (2S,4R)-glutamic acid di-t-butyl ester 5. 5 is then subjected to deprotection reaction with an appropriate agent such as hydrogenation using a metal catalyst such as Pd/C to generate the final product (2S,4R)-glutamic acid di-t-butyl ester 6.

III. Prodrug Conversion In Vivo

The ester bond in any of the disclosed prodrug compositions can be readily cleaved in vivo. Therefore, prodrug compositions of Formula I can be hydrolyzed to the corresponding acids form in plasma. For example, (2S,4R)-4-methyl glutamic acid dimethyl ester (2) was readily hydrolyzed to generate (2S,4R)-4-methyl glutamic acid (1). Prodrug forms were shown to have enhanced analgesic effects.

IV. Pharmaceutical Compositions and Therapeutic Applications based on In vitro and In vivo studies

Although prodrugs will generally not be active in some of the following assays (because the is not yet converted to active form), the active form of the compounds can be assessed. Accordingly, reference to “compound” in the following assays encompasses use of the active form of the disclosed prodrug compounds.

Receptor Binding

The basic discovery described herein is of prodrugs of a class of compounds that selectively bind at the kainate receptor and modulate kainate receptor function. Binding of the ultimate compounds (that is the compounds resulting after conversion of the prodrug) can be determined using standard techniques. Modulation of the kainate EAA receptor, as demonstrated by compounds showing potent in vitro affinity for the kainate receptor, make the compounds useful for treating human neuropsychopharmacological conditions related to EAAs.

The following analytical methods were used to determine the binding for each ligand and are identified by the literature reference where each is more fully set forth:

N-methyl-D-aspartate (NMDA) Receptor Binding Assay with CGS 19755:

Murphy, et al. “Binding of

[³H]-3 -(2-carboxypiperazin-4-yl-D-propyl- 1 -phosphonic acid) to rat brain membranes: A selective, high affinity ligand of N-methyl-D-aspartate receptors” J. Pharm. Exper. Therapeutics. 240:778-784 (1987).

AMPA Receptor Binding Assay:

Murphy, et al. “Characterization of quisqualate recognition sites in rat brain tissue using [³H]alpha-amino-3-hydroxy-5-methylisoxazole-4-propionic acid and a filtration assay” Neurochemical Research. 12:775-781 (1987).

Kainate Receptor Binding Assay with Kainate:

London, et al. “Specific binding of [³H]kainate to receptor sites in rat brain”. Molecular Pharmacology. 15:492-5-5 (1979).

In Vitro Assays of Physiological Activity and Potency

In combination, in vitro and in vivo assays are predictive of the activity of the disclosed compounds for treatment of patients. This is supported, for example, by U.S. Pat. No. 5,061,721 to Cordi et al. on the use of a combination of D-cycloserine and D-alanine to treat Alzheimer's disease, age-associated memory impairment, learning deficits, and psychotic disorders, as well as to improve memory or learning in healthy individuals, and U.S. Pat. No. 5,086,072 to Trullas et al. on 1-aminocyclo-propane carboxylic acid (ACPC), which modulates the NMDA receptor. ACPC and its derivatives can be used to treat neuropharmacological disorders resulting from excessive activation of the NMDA receptor, such as occurs in ischemia. NMDA antagonists and partial agonists have clearly been shown to be useful in human clinical trials based on in vitro and in vivo assays, as described by Hutchinson, et al., J. Med. Chem. 32, 2171-2178 (1989). Hutchinson, et al., (1989) reported that 4-(phosphonomethyl)-2-piperidine carboxylic acid (CGS-19755), a competitive glutamate antagonist for the NMDA receptor, is active in animal models of neurodegenerative diseases such as stroke and is currently undergoing human clinical evaluation for the treatment of strokes and head trauma.

The following tests are used to demonstrate that binding activity correlates with physiological activity, both in vitro and in vivo. The results of these tests indicate that kainate antagonists and partial agonists will be effective clinically for treatment of a variety of disorders, including includes cognitive, learning, and memory deficits, chemical toxicity (including substance tolerance and addiction), excitotoxicity, neurodegenerative disorder (such as Huntington's disease, Parkinson's disease, and Alzheimer's disease), post-stroke sequelae, epilepsy, seizures, mood disorders (such as bipolar disorder, dysthymia, and seasonal effective disorder), depression, and pain. Neurodegenerative disorders can result from dysfunction or malfunction of the receptor.

A. Electrophysiology

Tissue slice or whole cell electrophysiology as described by Yamada (Neurophysiology, 1994 and references therein) is used to measure agonist, partial agonist, or antagonist properties of drugs for glutamate receptors. This is a useful assay to demonstrate the in vivo activity of compounds formed from the prodrugs described herein, since it is predictive of efficacy, defined as the potency of the compound. This is distinct from the binding affinity.

For example, whole cell electrophysiology shows that (2S,4R)-4-methyl glutamic acid (which results from conversion of an appropriate prodrug as disclosed herein), modulates the kainate but not the AMPA receptor. Rat GluR6 kainate receptors were expressed in HEK 293 cells in culture and evaluated by the patch clamp technique. (2S,4R)-4-Methyl glutamic acid at doses of 10 nM to 20 μM completely and reversibly blocked the current evoked by 300 mM kainate.

The following assay can also be used to evaluate the physiological activity and potency of the prodrug compounds described herein.

B. Rat Mechano-allodynia Pain Model

This test is to determine the extent of protection by a test compound to neuropathic pain sensations. With the rat standing on an elevated perforated floor, mechano-allodynia is measured by applying from beneath a graded series of von Frey hairs to the mid-plantar region of the effected paws. The hair that evokes at least one withdrawal response is designated the threshold level when compared to the sham treated nerve. Alternatively, the paw can be illuminated with a noxious radiant heat and the time to paw withdrawal is measured.

The model is described by Bennett, Neuro. Report 5, 1438-1440 (1994), and references cited therein. He measures changes in withdrawal latency after chronic constriction injury; a rat is prepared by bilaterally exposing the sciatic nerves on both thighs. On one side, loosely fitting constrictive ligatures are tied around the nerve; the other side is sham manipulated but not ligated. The model can also be used to measure increases in sensitivity and decreases in latency after injection of an irritating or pain inducing substance such as capsaicin or carrageenan.

(2S,4R)-4-methyl glutamic acid (the active form of the disclosed prodrug compounds) was tested in the allodynia model after chronic constriction injury. A total of 40 adult male Sprague-Dawley rats, 250-300 g, were used. Baseline measures of withdrawal responses evoked by heat and mechanical stimuli were determined for each hind paw and obtained for at least 4 consecutive days until withdrawal and latency responses were stable. Four groups of 10 rats each were sorted to establish equivalent baseline mean responses to heat and mechanical stimuli. Each group was randomly assigned to 1 of 4 treatment conditions: vehicle, 10, 50, or 100 mg/kg compound i.p. On test day, withdrawal responses to heat and mechanical stimuli were measured before intraperitoneal injection of the treatment condition. (2S,4R)-4-methyl glutamic acid (the active form of the disclosed prodrug compounds) was also tested in the allodynia model after injection of capsaicin. Capsaicin (10 μl of 0.1%, intraplantar) was administered 30 minutes after i.p. injection. Withdrawal responses to heat and mechanical stimuli were again measured 5 and 30 minutes post capsaicin. The contralateral paw served as an internal control. The experimenter was blind to the treatment condition.

(2S,4R)-4-methyl glutamic acid was effective in blocking mechanical hyperalgesia at doses 10, 50, and 100 mg/kg. At the 5 minutes time point all compound doses were significantly different from vehicle (p<0.05). 30 minutes post capsaicin, only the 100 mg/kg group remained significantly different from vehicle (p<0.05). (2S,4R)-4-methyl glutamic acid was also effective in blocking hyperalgesia to heat. All three doses were significantly different from vehicle (p<0.05). By the 30 minute time point groups were not significantly different from vehicle.

The disclosed prodrug compositions are effective in preventing the development of hyperalgesia. The prodrugs were tested in the allodynia model after inducing pain with capsaicin. For example, doses of (2S,4R)-4-methyl glumatic acid dimethyl ester (2) i.p. of 0.1, 1, 10, 50 mg/kg were administered at 1 hour before the administering of capsaicin. The test showed that 10 and 50 mg/kg totally prevented the development of hyperalgesia. While the 0.1 mg/kg dose showed no effect, the 1 mg/kg dose had a modest effect of about 40% of hyperalgesia prevention.

A total of 24 adult male Sprague-Dawley rats, 250-300 g., were used and treated according to protocol described above using (2S,4R)-4-methyl glumatic acid methyl ester. The concentrations of the compound were 0, 0.1, 1, 10 or 50 mg/kg i.p. Hyperalgesia to mechanical stimuli and to radiant heat occurred immediately following capsaicin. However, at 10 mg/kg of the compound hyperalgesia to mechanical stimuli and to radiant heat was reduced (p<0.05).

A total of 33 adult male Sprague-Dawley rats, 250-300 g., were treated according to protocol described above using (2S,4R)-4-methyl glumatic acid ethyl ester. The concentrations of the compound were 0, 0. 1, 10, or 50 mg/kg i.p. The ethyl ester was effective in blocking mechanical hyperalgesia at doses 10 and 50 mg/kg (p<0.05), but was not effective in blocking hyperalgesia to heat.

In summary, the prodrugs showed activity at 10 mg/kg versus 50 mg/kg for the parent compound. Their activity also lasted up to an hour instead of up to 30 minutes.

V. Dosage Forms

A. Effective Dosage Ranges

The prodrug compounds described herein can be administered parenterally, either subcutaneously, intramuscularly, or intravenously, or alternatively, administered orally in a dose range of between approximately 0.5 mg/kg body weight and 150 mg/kg body weight.

B. Carriers and Additives

The disclosed prodrugs can be administered parenterally, in sterile liquid dosage forms. In general, water, a suitable oil, saline, aqueous dextrose, and related sugar solutions and glycols such as propylene glycol or polyethylene glycols are suitable carriers for parenteral solutions. Solutions for parenteral administration preferably contain a water soluble form of the active ingredient, suitable stabilizing agents, and, if necessary, buffer substances.

Antioxidizing agents such as sodium bisulfite, sodium sulfite, or ascorbic acid, either alone or in combination, can be used as suitable stabilizing agents. Also used are citric acid and its salts and sodium EDTA. In addition, parenteral solutions can contain preservatives, such as benzalkonium chloride, methyl- or propylparaben, and chlorobutanol.

The disclosed prodrugs can be administered orally in solid dosage forms, such as capsules, tablets and powders, or in liquid dosage forms, such as elixirs, syrups, and suspensions. Gelatin capsules contain the active ingredient and powdered carriers, such as lactose, starch, cellulose derivatives, magnesium stearate, or stearic acid. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere.

Other agents that can be used for delivery include liposomes, microparticles (including microspheres and microcapsules), and other release devices and forms that provide controlled, prolonged or pulsed, delivery or which enhance passage through the blood brain barrier, for example.

Bioerodible microspheres can be prepared using any of the methods developed for making microspheres for drug delivery, for example, as described by Mathiowitz and Langer, J. Controlled Release 5:13-22 (1987); Mathiowitz, et al., Reactive Polymers 6:275-283 (1987); and Mathiowitz, et al., J. Appl. Polymer Sci. 35, 755-774 (1988), the teachings of which are incorporated herein. The selection of the method depends on the polymer selection, the size, external morphology, and crystallinity that is desired, as described, for example, by Mathiowitz, et al., Scanning Microscopy 4:329-340 (1990); Mathiowitz, et al, J. Appl. Polymer Sci. 45, 125-134 (1992); and Benita, et al., J. Pharm. Sci. 73:1721-1724 (1984), the teachings of which are incorporated herein. Methods routinely used by those skilled in the art include solvent evaporation, hot melt encapsulation, solvent removal, spray drying, phase separation and ionic crosslinking of gel-type polymers such as alginate or polyphosphazines or other dicarboxylic polymers to form hydrogels.

Other delivery systems including films, coatings, pellets, slabs, and devices can be fabricated using solvent or melt casting, and extrusion, as well as standard methods for making composites.

The microparticles can be suspended in any appropriate pharmaceutical carrier, such as saline, for administration to a patient. In the most preferred embodiment, the microparticles will be stored in dry or lyophilized form until immediately before administration. They will then be suspended in sufficient solution for administration. The polymeric microparticles can be administered by injection, infusion, implantation, orally, or administration to a mucosal surface, for example, the nasal-pharyngeal region and/or lungs using an aerosol, or in a cream, ointment, spray, or other topical carrier, for example, to rectal or vaginal areas. The other devices are preferably administered by implantation in the area where release is desired. The materials can also be incorporated into an appropriate vehicle for transdermal delivery as well as stents. Appropriate vehicles include ointments, lotions, patches, and other standard delivery means.

The compositions and methods of use thereof disclosed herein can be further understood by the following non-limiting examples.

### EXAMPLES

### Example 1

### Synthesis of (2S,4R)-4-Methyl Glutamic Acid Dimethyl Ester Hydrochloride (2)

(2S,4R)-4-Methyl glutamic acid (1, 1.81 g, 11.2 mmol) was dissolved in methanol and cooled to 0° C. Thioyl chloride (2.75 g, 23.1 mmol) was added dropwise. After the addition, the reaction mixture was stirred at room temperature overnight. The solvent was evaporated, and the crude product was recrystallized with acetone to give 2 as a white crystal. The identification and characterization data for 2 are: m.p. 138-139° C.; MS (CI-NH₃): 190 (MH⁺); ¹HNMR (CDCl₃): 8.77 (b.s., 3H), 4.28 (b.s., 1H), 3.82 (s, 3H), 3.70 (s, 3H), 2.98-2.91 (m, 1H), 2.50-2.40 (m, 1H), 2.21-2.12 (m, 1H), 1.28 (d, 3H, J=7.1 Hz).

### Example 2

### Synthesis of (2S,4R)-4-Methyl Glutamic Acid Diethyl Ester Hydrochloride (3)

(2S,4R)-4-Methyl glutamic acid (1, 450 mg, 2.79 mmol) was dissolved in ethanol and cooled to 0° C. Thioyl chloride (815 mg, 6.85 mmol) was added dropwise. After the addition, the reaction mixture was stirred at room temperature overnight. The solvent was evaporated and the crude product was recrystallized with acetone to give 3 as a white crystal. The identification and characterization data for 3 are: m.p. 158-160° C.; MS (CI-NH₃): 218 (MH⁺); ¹ HNMR (CDCl₃): 8.20 (b.s., 3H), 3.85-3.70 (m, 4H), 2.86-1.94 (m, 4H), 1.49-1.26 (m, 6H), 1.28 (d, 3H, J=7.1 Hz).

### Example 3

### Synthesis of (2S,4R)-N-Benzyloxycarbonyl-4-Methyl Glutamic Acid (4)

(2S,4R)-4-Methyl glutamic acid trifluoroacetic acid salt (1•TFA, 678 mg, 2.5 mmol) was dissolved in 2 N NaOH (5 mL). Benzyl chloroformate (427 mg, 2.5 mmol) was added. After the addition, the reaction mixture was stirred at room temperature overnight. The reaction mixture was adjusted to pH5 by adding 1 N HCl, and extracted with AcOEt (2×10 mL). The combined AcOEt phases were dried over MgSO₄. After the evaporation of solvent, 4 (300 mg, 40%) was obtained as a light yellow syrup which was used for next step without further purification.

### Example 4

### Synthesis of (2S,4R)-N-Benzyloxycarbonyl-4-Methyl Glutamic Acid Di-tert-butyl Ester (5)

Compound 4 (300 mg, 1.02 mmol) was dissolved in CH₂Cl₂ (5 mL), then O-tert-butyl-N,N′-diisopropylisourea (421 mg, 2.1 mmol) was added. After the addition, the reaction mixture was stirred at room temperature overnight. The reaction mixture was filtered through a Celite pad, and washed with 1 N HCl (10 mL), 1 N NaOH (10 mL), and water (10 mL), dried over MgSO₄. After the evaporation of solvent, 5 (84 mg, 20%) was obtained as a light yellow syrup which was used for next step without further purification.

### Example 5

### Synthesis of (2S,4R)-4-Methyl Glutamic Acid Di-tert-butyl Ester (6)

Compound 5 (84 mg, 1.02 mmol) was dissolved in AcOEt (5 mL), then 10% Pd-C (10 mg) was added. The reaction mixture was stirred at room temperature under hydrogen and monitored with TLC. After the reaction complete, the reaction mixture was filtered through a Celite pad. After the evaporation of solvent, 6 (30 mg, 50%) was obtained as a light yellow syrup. The identification and characterization data for 6 are: MS (CI-NH₃): 274 (MH⁺); ¹HNMR (CDCl₃): 8.0 (b.s., 2H), 3.8-3.60 (m, 4H), 1.52 (s, 9H), 1.50 (s, 9H), 1.29 (d, 3H, J=7.1 Hz).

### Example 6

### Synthesis of (2R,4S)-4-Methyl Glutamic Acid Dimethyl Ester Hydrochloride (8)

(2R,4S)-4-Methyl glutamic acid (7, 1.81 g, 11.2 mmol) was dissolved in methanol and cooled to 0° C. Thioyl chloride (2.75 g, 23.1 mmol) was added dropwise. After the addition, the reaction mixture was stirred at room temperature overnight. The solvent was evaporated and the crude product was recrystallized with acetone to give 8 as a white crystal. The identification and characterization data for 8 are: m.p. 138-139° C.; MS (CI-NH₃): 190 (MH⁺); ¹HNMR (CDCl₃): 8.77 (b.s., 3H), 4.28 (b.s., 1H), 3.82 (s, 3H), 3.70 (s, 3H), 2.98-2.91 (m, 1H), 2.50-2.40 (m, 1H), 2.21-2.12 (m, 1H), 1.28 (d, 3H, J=7.1 Hz).

### Example 7

### Synthesis of (2R,4S)-4-Methyl Glutamic Acid Diethyl Ester Hydrochloride (9)

(2R,4S)-4-Methyl glutamic acid (7, 450 mg, 2.79 mmol) was dissolved in ethanol and cooled to 0° C. Thioyl chloride (815 mg, 6.85 mmol) was added dropwise. After the addition, the reaction mixture was stirred at room temperature overnight. The solvent was evaporated and the crude product was recrystallized with acetone to give 9 as a white crystal. The identification and characterization data for 9 are: m.p. 158-160° C; MS (CI-NH₃): 218 (MH⁺); ¹HNMR (CDCl₃): 8.20 (b.s., 3H), 3.85-3.70 (m, 4H), 2.86-1.94 (m, 4H), 1.49-1.26 (m, 6H), 1.28 (d, 3H, J=7.1 Hz).

### Example 8

### Synthesis of (2R,4S)-N-Benzyloxycarbonyl-4-Methyl Glutamic Acid (9)

(2R,4S)-4-Methyl glutamic acid trifluoroacetic acid salt (7•TFA, 678 mg, 2.5 mmol) was dissolved in 2 N NaOH (5 mL). Benzyl chloroformate (427 mg, 2.5 mmol) was added. After the addition, the reaction mixture was stirred at room temperature overnight. The reaction mixture was adjusted to pH5 by adding 1 N HCl, and extracted with AcOEt (2×10 mL). The combined AcOEt phases were dried over MgSO₄. After the evaporation of solvent, 10 (300 mg, 40%) was obtained as a light yellow syrup which was used for next step without further purification.

### Example 9

### Synthesis of (2R,4S)-N-Benzyloxycarbonyl-4-Methyl Glutamic Acid Di-tert-butyl Ester (11)

Compound 10 (300 mg, 1.02 mmol) was dissolved in CH₂Cl ₂ (5 mL), then O-tert-butyl-N,N′-diisopropylisourea (421 mg, 2.1 mmol) was added. After the addition, the reaction mixture was stirred at room temperature overnight. The reaction mixture was filtered through a Celite pad, and washed with 1 N HCl (10 mL), 1 N NaOH (10 mL), and water (10 mL), dried over MgSO₄. After the evaporation of solvent, 11 (84 mg, 20%) was obtained as a light yellow syrup which was used for next step without further purification.

### Example 10

### Synthesis of (2R,4S)-4-Methyl Glutamic Acid Di-tert-butyl Ester (12)

Compound 11 (84 mg, 1.02 mmol) was dissolved in AcOEt (5 mL), then 10% Pd-C (10 mg) was added. The reaction mixture was stirred at room temperature under hydrogen and monitored with TLC. After the reaction complete, the reaction mixture was filtered through a Celite pad. After the evaporation of solvent, 12 (30 mg, 50%) was obtained as a light yellow syrup. The identification and characterization data for 12 are: MS (CI-NH₃): 274 (MH⁺); ¹HNMR (CDCl₃): 8.0 (b.s., 2H), 3.8-3.60 (m, 4H), 1.52 (s, 9H), 1.50 (s, 9H), 1.29 (d, 3H, J=7.1 Hz).

Examples shown in Table 1 were prepared or can be prepared by the methods outline in Schemes I-IV presented above and procedures described in the Examples using the appropriate starting materials and reagents.

TABLE 1
Exemplary Compounds of Formula 1

| | | | | | | Stereo |
|---|---|---|---|---|---|---|
| Ex. | R¹ | R² | R³ | R⁴ | R⁵, R⁶ | Config. |
| 1 | —CH₃ | H | H | H | —CH₃ | 2S, 4R |
| 2 | —CH₃ | H | H | H | —CH₂CH₃ | 2S, 4R |
| 3 | —CH₃ | H | H | Cbz | H | 2S, 4R |
| 4 | —CH₃ | H | H | Cbz | t-Bu | 2S, 4R |
| 5 | —CH₃ | H | H | H | t-Bu | 2S, 4R |
| 6 | —CH₃ | H | H | H | —CH₃ | 2R, 4S |
| 7 | —CH₃ | H | H | H | —CH₂CH₃ | 2R, 4S |
| 8 | —CH₃ | H | H | Cbz | H | 2R, 4S |
| 9 | —CH₃ | H | H | Cbz | t-Bu | 2R, 4S |
| 10 | —CH₃ | H | H | H | t-Bu | 2R, 4S |

Those skilled in the art will recognize, or be able to ascertain using no more than routine experimentation, many equivalents to the specific embodiments of the invention described herein. Such equivalents are intended to be encompassed by the following claims.

We claim:

1. An alkyl carboxy amino acid ester compound having the following formula: 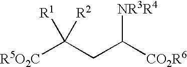
wherein
R¹, R², R⁵ and R⁶ are independently
1) C1-C6-alkyl,
2) C3-C4-alkenyl,
3) C3-C5-cycloalkyl;
R³ and R⁴ are independently
1) H
2) C1-C6-alkyl,
3) C3-C4-alkenyl,
4) C3-C5-cycloalkyl,
5) C1-C6-alkyl-CO—
6) C1-C6-alkyl-OCO—
7) C1-C6-alkyl-NHCO—
8) HCO—, or
9) C3-C6-alkynyl;
R³ and R⁴ taken together can be —CH₂(CH₂)ₙCH₂—;
n is 0-3;
and pharmaceutically acceptable salts of these compounds.

2. The compound of claim 1 wherein
R¹ is CH₃;
wherein R² is H;
wherein R³ and R⁴ are independently
1) H
2) C1-C6-alkyl,
3) C3-C4-alkenyl,
4) C3-C5-cycloalkyl,
5) C1-C6-alkyl-CO—,
6) C1-C6-alkyl-OCO—,
7) C1-C6-alkyl-NHCO—,
8) HCO—, or
R³ and R⁴ taken together can be —CH₂(CH₂)ₙCH₂— in which n is an integer selected from the group consisting of 0, 1, 2 and 3;
and wherein R⁵ and R⁶ are independently
1) C1-C6-alkyl,
2) C3-C4-alkenyl, or
3) C3-C5-cycloalkyl;
and pharmaceutically acceptable salts of these compounds.

3. The compound of claim 2 wherein
R³ and R⁴ taken together can be —CH₂(CH₂)ₙCH₂— in which n is an integer selected from the group consisting of 0, 1, 2 and 3;
and pharmaceutically acceptable salts of these compounds.

4. The compound of claim 1 selected from the group consisting of:
(2S,4R)-4-methyl glutamic acid dimethyl ester;
(2S,4R)-4-methyl glutamic acid diethyl ester;
(2S,4R)-4-methyl glutamic acid di-tert-butyl ester;
(2R,4S)-4-methyl glutamic acid dimethyl ester;
(2R,4S)-4-methyl glutamic acid diethyl ester; and
(2R,4S)-4-methyl glutamic acid di-tert-butyl ester.

5. A pharmaceutical composition comprising
a compound for selectively modulating ion flow through the kainate receptor in combination with a pharmaceutically acceptable carrier for administration to a patient in need thereof,
wherein the compound is an alkyl carboxy amino acid compounds having the formula: 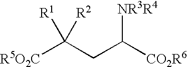
wherein
R¹, R², R5 and R⁶ are independently
1) C1l-C6-alkyl,
2) C3-C4-alkenyl,
3) C3-C5-cycloalkyl;
R³ and R⁴ are independently
1) H
2) C1-C6-alkyl,
3) C3-C4-alkenyl,
4) C3-C5-cycloalkyl,
5) C1-C6-alkyl-CO—
6) C1-C6-alkyl-OCO—
7) C1-C6-alkyl-NHCO—
8) HCO—, or
9) C3-C6-alkynyl;
R³ and R⁴ taken together can be —CH₂(CH₂)ₙCH₂—;
n is 0-3;
and pharmaceutically acceptable salts of these compounds.

6. The composition of claim 5 wherein
R¹ is CH₃;
wherein R² is H;
wherein R³ and R⁴ are independently
1) H
2) C1-C6-alkyl,
3) C3-C4-alkenyl,
4) C3-C5-cycloalkyl,
5) C1-C6-alkyl-CO—,
6) C1-C6-alkyl-OCO—,
7) C1-C6-alkyl-NHCO—,
8) HCO—, or
R³ and R⁴ taken together can be —CH₂(CH₂)ₙCH₂— in which n is an integer selected from the group consisting of 0, 1, 2 and 3;
and wherein R⁵ and R⁶ are independently
1) C1-C6-alkyl,
2) C3-C4-alkenyl, or
3) C3-C5-cycloalkyl;
and pharmaceutically acceptable salts of these compounds.

7. The composition of claim 6 wherein
R³ and R⁴ taken together can be —CH₂(CH₂)ₙCH₂—in which n is an integer selected from the group consisting of 0, 1, 2 and 3;
and pharmaceutically acceptable salts of these compounds.

8. The composition of claim 5 selected from the group consisting of:
(2S,4R)-4-methyl glutamic acid dimethyl ester;
(2S,4R)-4-methyl glutamic acid diethyl ester;
(2S,4R)-4-methyl glutamic acid di-tert-butyl ester;
(2R,4S)-4-methyl glutamic acid dimethyl ester;
(2R,4S)-4-methyl glutamic acid diethyl ester; and
(2R,4S)-4-methyl glutamic acid di-tert-butyl ester.
and pharmaceutically acceptable salts of these compounds.

9. A method for treating a patient having a disorder associated with excessive or insufficient activation of the kainate subtype of the ionotropic EAA receptors comprising administering to the patient an effective amount of the pharmaceutical composition of claim 5 to alleviate the symptoms of the disorder.

10. The method of claim 9 wherein the disorder is selected from the group consisting of neurological, neuropsychological, neuropsychiatric, neurodegenerative, neuropsychopharmacological and functional disorders.

11. The method of claim 9 wherein the disorder is pain comprising administering to the patient an effective amount of the pharmaceutical composition to alleviate the pain.

12. The method of claim 9 wherein the disorder is selected from the group of cognitive disorders associated with deactivation, suboptimal activation, and over-activation of the kainate receptor.

13. The method of claim 9 wherein the disorder is a decrease or loss of memory, learning, or associated mental processes comprising administering to the patient an effective amount of the pharmaceutical composition to enhance or increase cognition.
